(19) United States
(12) Patent Application Publication
(10) Pub. No.: US 2018/0078641 A1
(43) Pub. Date: Mar. 22, 2018

(54) Method for treating infectious diseases using emissive energy

(71) Applicant: Marv Enterprises, LLC, Hermitage, PA (US)

(72) Inventors: Mitchell S. Felder, Hermitage, PA (US); Danny W. Muse, Huntsville, AL (US)

(21) Appl. No.: 15/811,809

(22) Filed: Nov. 14, 2017

Related U.S. Application Data
(63) Continuation of application No. 15/305,344, filed on Oct. 19, 2016, now abandoned.
(60) Provisional application No. 61/987,754, filed on May 2, 2014.

(51) Int. Cl. A61K 41/00; G01N 33/58; A61M 1/36; A61K 49/00

(52) U.S. Cl. CPC A61K 41/0019; G01N 2469/10; G01N 33/582; A61M 1/3686; A61M 1/362; A61K 49/0058

(57) ABSTRACT

The present invention relates to the treatment of infectious diseases, specifically by extracorporeally eradicating the pathogen. This invention comprises methods for the extracorporeal treatment of infectious diseases that will remove infectious pathogens (leukemia cells, bacteria, viruses, or fungi causing a septicemia, metastatic cancer cells, target protein, viruses, parasites, fungi and prions) in humans by targeting such pathogens with a laser or other high-energy source of emissive radiation. More specifically, the method involves removing a bodily fluid from a patient, attaching an antibody to pathogens in the bodily fluid, sensing the antibody-pathogen moiety, using a high-powered, focused laser, or other suitable light source, to destroy the antibody-pathogen moiety, removing the remains of the antibody-pathogen by filtering or other suitable mechanism(s), and returning the bodily fluid to the patient.

### CROSS-REFERENCE TO RELATED APPLICATIONS

This application claims benefit under 35 U.S.C. §119(e) of U.S. Patent Application No. 61/987,754, filed May 2, 2014, which is hereby incorporated herein by reference in its entirety.

### FIELD OF THE INVENTION

The present invention relates to the treatment of infectious diseases.

### BACKGROUND OF THE INVENTION

Approximately one out of three people in the world will die of an infectious disease. Infectious diseases have greatly influenced world history. Major historical military campaigns have been reversed by the outbreak of diseases, such as typhus and dysentery. Yersinia pestis, the black plague, profoundly altered the course of medieval European history.

Infectious diseases are caused by pathogenic microorganisms, such as bacteria, viruses, parasites or fungi; the diseases can be spread, directly or indirectly, from one person to another. Zoonotic diseases are infectious diseases of animals that can cause disease when transmitted to humans.

Today, four of the top ten leading causes of death are infectious diseases in low- and middle-income countries. The African region is a region of the world in which communicable diseases still dominate as the main cause of death. The World Health Organization is prioritizing an intensified global commitment to safeguard antibiotics for preventing and controlling infectious diseases. Growing resistance by microbes to antibiotics threatens the continued effectiveness of many medicines.

### SUMMARY OF THE INVENTION

It is a primary object of this invention to provide a method of treating infectious diseases by extracorporeally eradicating the pathogen.

Specifically, it is an object of this invention to provide a method of extracorporeal treatment of infectious diseases that will remove infectious pathogens (leukemia cells, bacteria, viruses, or fungi causing a septicemia, metastatic cancer cells, target protein, viruses, parasites, fungi and prions) in humans by targeting such pathogens with a laser or other high energy source of emissive radiation.

More specifically, the method of the present invention involves removing a bodily fluid from a patient, attaching an antibody to pathogens in the bodily fluid, sensing the antibody-pathogen moiety, using a high-powered, focused laser to destroy the antibody-pathogen moiety, removing the remains of the antibody-pathogen, by filtering or another suitable mechanism, and returning the bodily fluid to the patient.

### BRIEF DESCRIPTION OF THE DRAWINGS

FIG. 1 is a partial cross sectional view of a cylinder and tubing used to deliver a treatment to a bodily fluid.

FIG. 2 is a partial cross sectional view showing additional detail of the cylinder and tubing of FIG. 1.

FIG. 3 is a schematic illustrating the method of the present invention.

FIG. 4. Digital micromirror device. Image source: www.opli.net

FIG. 5. SEM image of a vectored mirror in a micromirror device. Source: Texas Instruments (1987).

FIG. 6. Example of a wide channel flow cuvette with sensing area emphasized.

FIG. 7. Simplified diagram of an embodiment of the present invention. Typical optical components have been omitted for clarity.

FIG. 8. Simplified “Sense, Process, and Annihilate” illustration. (a) Multiple pathogens observed through fluorescence emissions. (b) Bitmap is generated with target position. (c) Image is compared to previous images to predict future position of pathogens. (d) Bitmap with future positions is generated and loaded into the digital micromirror device. (e) Intense photonic energy is projected to target areas.

### DETAILED DESCRIPTION OF THE INVENTION

The present invention relates to a method of treating infectious diseases in a patient's body fluid extracorporeally for the purpose of removing, by filtering or dialysis, targeted pathogens (bacteria, viruses, parasites, fungi, or prions) from body fluids using a short-duration pulse-beam from a laser or other high energy radiation emissive source.

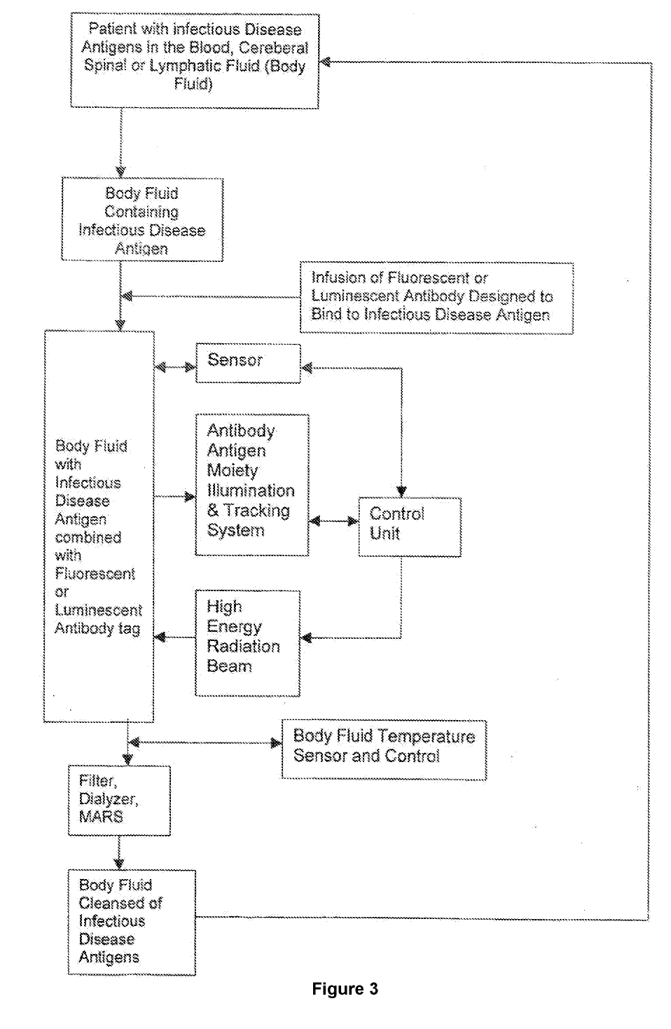

The method of the invention is best described with reference to FIG. 3. The invention includes several stages.

A body fluid (e.g., blood, CSF, or lymphatic fluid) is withdrawn from a patient using standard medical techniques.

In the first stage a treatment is applied to a body fluid extracorporeally. The treatment comprises a fluorescent or luminous tagged antibody (F/LT Ab) directed at the targeted pathogenic antigen (TPA). As used herein, the acronym “TPA” includes bacteria, virus, parasite, fungus, or prion. This forms a fluorescently tagged or luminously tagged antibody/targeted pathogen antigen complex (F/LT Ab-TPA complex).

The second stage includes the substantial elimination of the treatment from the extracorporeal body fluid using a laser.

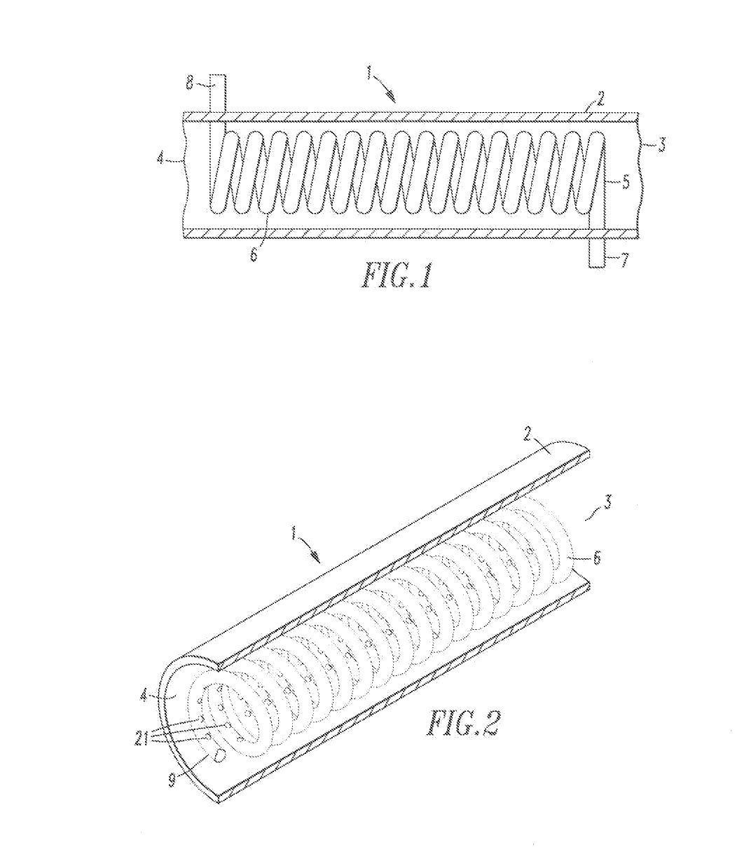

As shown in FIG. 1, the first stage can include an exterior wall to define a treatment chamber 5. The treatment conveniently can be applied in the treatment chamber 5. Residence times of the body fluid the first stage can be altered by changing the dimensions of the treatment chamber, or by using a dialysis vacuum pump. With reference to FIG. 1, body fluid enters the inlet 3, passes through the treatment chamber 5, and exits the outlet 4.

The treatment of an antibody with an attached fluorescently tagged moiety or luminescently tagged moiety (F/LT Ab) targeting the TPA can be applied from a delivery tube 6 located within the treatment chamber 5.

An inferior wall 9 defines the delivery tube 6. The delivery tube 6 can include at least one lead 7, 8. The lead 7, 8 can deliver the treatment to the treatment chamber 5. Conveniently, the delivery tubes 6 will have a high contact surface area with the body fluid. As shown, the delivery tube 6 comprises a helical coil.

With reference to FIG. 2, when the treatment includes the administration of a fluorescently tagged or luminescently tagged antibody (F/LT Ab), the delivery tube 6 can be hollow and the interior wall 9 can define a plurality of holes 21. The F/LT Abs can be pumped through the delivery tube 6 to achieve a desired concentration of F/LT Abs in the body fluid. The F/LT Abs perfuse through the holes 21. The delivery tube 6 can include any suitable material including, for example, metal, plastic, ceramic, or combinations thereof. The delivery tube 6 can also be rigid or flexible. In one embodiment, the delivery tube 6 is a metal tube perforated with a plurality of holes. Alternatively, the delivery tube 6 can be plastic.

The F/LT Abs, targeting the targeted pathogenic antigen (TPA: bacteria, virus, parasite, fungus, or prion) can be delivered in a concurrent or counter-current mode with reference to the flow of the body fluid. In counter-current mode, the body fluid enters the treatment chamber 5 at the inlet 3. The F/LT Ab-TPAs can enter through a first lead 8 near the outlet 4 of the treatment chamber 5. Body fluid then passes to the outlet 4 and the F/LT Ab-TPAs pass to the second lead 7 near the inlet 3.

As stated, the present invention relates to a method of treating infectious diseases in a patient's body fluid extracorporeally for the purpose of removing the TPA from body fluids. The process shown in FIG. 3 includes an illumination system, an optic or other suitable sensor for detecting individual F/LT Ab-TPAs, and a high energy radiation source, such as a laser or other coherent light beam. The body fluid is pumped past the sensor where the body fluid is illuminated and the F/LT Ab-TPAs are identified. The sensor is connected to a control unit. The signal from the sensed F/LT Ab-TPAs is transmitted to a control unit which controls a high energy emissive source. The receipt of a F/LT Ab-TPA signal causes the control unit to emit a short-duration pulse-beam from a laser or other high energy radiation emissive source. The energy of the emitted radiation annihilates the F/LT Ab-TPA, thereby destroying its disease-causing potential.

The second stage substantially eliminates, through laser or other high-energy radiation emissive source targeting and annihilating, the F/LT Ab-TPAs from the bodily fluid.

The body fluid is passed through a filter or dialysis device that selectively removes the F/LT Ab-TPA, thereby removing the remains of the disease-causing antigen. The filtering or dialysis removes the remains of the F/LT Ab-TPAs from the body fluid. This removal may be accomplished by any suitable means, including, for example, hemodialysis devices, molecular adsorbents recirculation system (MARS), single-pass albumin dialysis (SPAD), continuous veno-venous hemodiafiltration (CVVHDF), Fresenius Medical Care's Prometheus® process (a combination of albumin adsorption with high-flux hemodialysis after selective filtration of the albumin fraction through a specific polysulfone filter, AlbuFlow®), or other suitable detoxification technique.

The detoxified and cleaned body fluid is then returned to the patient, free of the infectious pathogen(s). The method includes two stages. The first stage includes a chamber for treating body fluids by infusing an antibody with an attached fluorescent or luminous tagged (F/LT) moiety directed to a specific pathogenic infectious antigen into the extracorporeal body fluid. A second stage receives the treated body fluid and includes a sensor for sensing the F/LT Ab-TPAs, and a laser or other high-energy emissive source of radiation for annihilating, destroying, or deactivating the F/LT ATPAs (fluorescent or luminous tagged antibody-targeted pathogen antigen complex).

Alternatively, target antigens (TAs: examples of which include leukemia cells, bacteria, viruses, or fungi causing a septicemia, metastatic cancer cells, target proteins) are captured using antibody microarrays containing fluorescent (Fl) or luminescent (Lu) antibodies (Fl-Ab/Lu-Ab) in microarrays. An antibody microarray is a protein microarray; a collection of capture antibodies are fixed on a solid surface, such as glass, plastic and silicon chip for the purpose of detecting antigens. Antibody microarrays are composed of millions of identical monoclonal antibodies attached at high density on glass or plastic slides.

During the extracorporeal exposure of the TAs, there is created an antibody pathogen complex of FI-Ab TA or Lu-Ab-TA on the microarray. The complexes are then tracked using an appropriate sensor and obliterated using a high energy focused radiation beam such as a laser.

The body fluid is then forced through a container constructed from a transparent material such as glass, or other material, which exposes the F/LT Ab-TPAs (fluorescent or luminous tagged antibody-targeted pathogen antigen complex) to a light-sensing device. The sensing device also creates an enlarged, magnified visual image of the F/LT Ab-TPAs (fluorescent or luminous tagged antibody-targeted pathogen antigen complex). A concentrated and focused intense energy beam, such as light, is then used to properly illuminate the F/LT Ab-TPAs within the body fluid.

A laser or other high-energy radiation emissive source is then used to annihilate the targeted F/LT Ab-TPAs (fluorescent or luminous tagged antibody-targeted pathogen antigen complex). The radiation source uses very short bursts of less than a millisecond to annihilate the F/LT Ab-TPAs. Each F/LT Ab-TPA is very rapidly identified and precisely located. The targeted F/LT Ab-TPAs are identified and tracked using optical or digital enhancement or magnification. The very rapid (0.0001 to 0.1 ms) location and tracking of each targeted F/LT Ab-TPA is achieved using computer graphics and computer programs well known in the art. An alternative methodology would use optical pattern recognition of the F/LT Ab-TPAs. The temperature of the treated body fluid is maintained at 98.6° F. via continuous cooling of the body fluid using a standard cooling apparatus. A constant thermostatic measurement and control system continuously monitors the process to maintain the body fluid temperature at 98.6° F.

A multiplicity of flow channels are used for locating and targeting the F/LT Ab-TPAs. The channels have a width of −1.0 mm to 0.0001 mm and a base length of −10 cm to −0.1 cm. The dimensions of the channels are predetermined according to the amount of body fluid (blood, CSF, or lymphatic fluid) to be treated. A vacuum pump is used to continuously pull the targeted body fluid volume, which is to be treated, at a predetermined speed, towards and through the location of the laser or other high-energy radiation emissive source treatment. Multiple passes of the body fluid through the channels may be used at the completion of this treatment process until all F/LT Ab-TPAs have been eliminated. The treated body fluid, which has been filtered or dialyzed and shown to be completely free of all the targeted pathogenic antigens is then returned to the patient via the catheter used.

An alternative methodology of the present intervention would use a designer fluorescent or luminous antibody (F/LT Ab) with an additionally attached macromolecular moiety (MM). The macromolecular moiety, attached to the fluorescent or luminous antibody, would be 1.000 mm to 0.005 mm in diameter. The fluorescent or luminous antibody-macromolecular moiety-targeted pathogen antigen complex (F/LT MM Ab-TPA) would then be blocked from reentering the patient's CSF and/or body fluid circulation, by using a filter or dialysis machine having a series of microscreens which contain openings with a diameter 50.0000% to 99.9999% less than the diameter of the designer antibody-macromolecular moiety (F/LT MM Ab). The microscreen opening(s) must have a diameter of at least 35 micrometers to allow for the passage and return to circulation of the non-pathological-inducing body fluid (blood, CSF or lymphatic fluid) constituents. A laser or other high-energy radiation emissive source is then used to annihilate the targeted fluorescent or luminous antibody-macromolecular moiety-targeted pathogen antigen complex (F/LT MM Ab-TPA). By using the macromolecular moiety the annihilation of the F/LT MM Ab-TPA is greatly simplified, with an extended location and tracking period.

Alternatively, the fluorescently tagged or luminously tagged antibody/targeted pathogen antigen complex (F/LT Ab-TPA complex) may be captured, by using antibody microarrays which contain antibodies to the F/LT Ab-TPAs. The antibody microarrays are composed of millions of identical monoclonal antibodies attached at high density on glass or plastic slides. After sufficient extracorporeal exposure of the F/LT Ab-TPAs to the antibody microarrays, the antibody microarrays may be disposed of, using standard medical practice. Alternatively, the laser may be utilized to annihilate the F/LT Ab-TPA complexes which had been captured by the antibody microarrays. By using the antibody microarrays the annihilation of the F/LT Ab-TPA is greatly simplified, with an extended location and tracking period.

Another alternative methodology of the present invention comprises positioning the F/LT Ab-TPA by using a designer antibody containing an iron (Fe) moiety. This will then create a Fe-F/LT Ab-TPA complex (iron-fluorescent or luminous antibody-targeted pathogenic antigen complex). This iron containing complex may then be efficaciously positioned for annihilation by using a strong, localized magnetic force field. By using the Iron (Fe) moiety, the annihilation of the F/LT Ab-TPA is greatly simplified, with an extended location and tracking period. When there is a return of body fluid into the patient, the addition of standard medications, such as penicillin may be used to help prevent reinfection by any remaining pathogens.

A treatment of body fluid would involve 15-1500 cc of body fluid during a standard treatment procedure. The frequency of such treatments would depend upon the underlying symptomatology and pathology of the patient, and would be determined by the patient's physician.

Many examples exist in literature whereby radiant light energy, from a laser or other source, has shown to be effective in annihilating blood borne pathogens. Methods described are typically applied to small-scale, low-volume samples of whole blood or serum, and are often used in the course of analysis or fundamental research rather than practical treatment of the associated diseases.

Challenges to such treatments include: 1) achieving high specificity of the targeted pathogen, thus minimizing damage to normal blood components such as erythrocytes, leukocytes and platelets, 2) processing infected blood at rates sufficient to be of practical use, and 3) providing effective treatment with robust, yet affordable equipments.

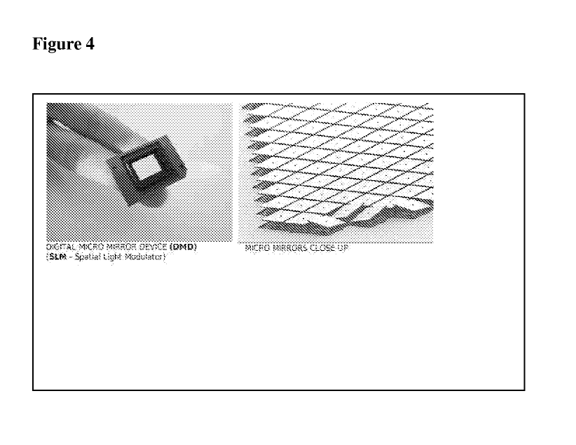

To address these challenges, and to reduce this novel treatment to practice, a method and apparatus are proposed making use of contemporary digital light processing (DLP) to annihilate blood borne pathogens. DLP is a technology developed by Texas Instruments and introduced in the late 1980s. DLP technology uses a silicon-based digital micromirror device (DMD), as shown in FIG. 4, to selectively project light onto a target, typically a screen, in a digital manner.

The DMD is an array of about several hundred thousand to over one million micron-scale mirrors that can be individually vectored, modulating intense light sources to produce bright, high-resolution images and video. Individual mirrors in a typical DMD are about 8 μm across, similar in size to many parasites and bacteria.

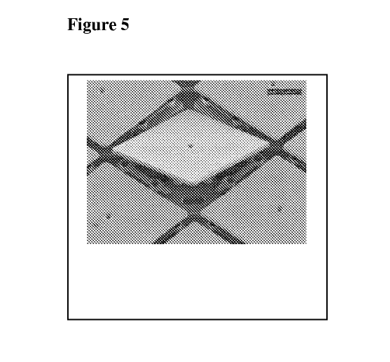

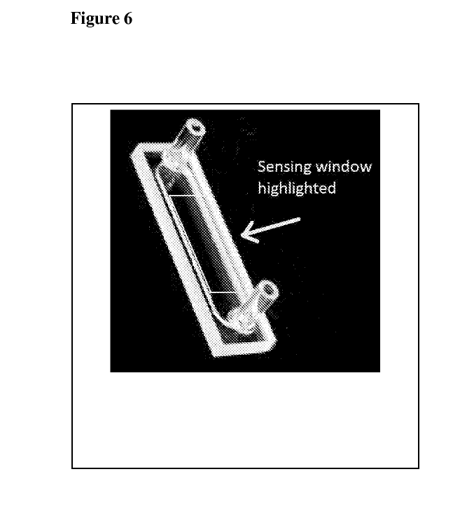

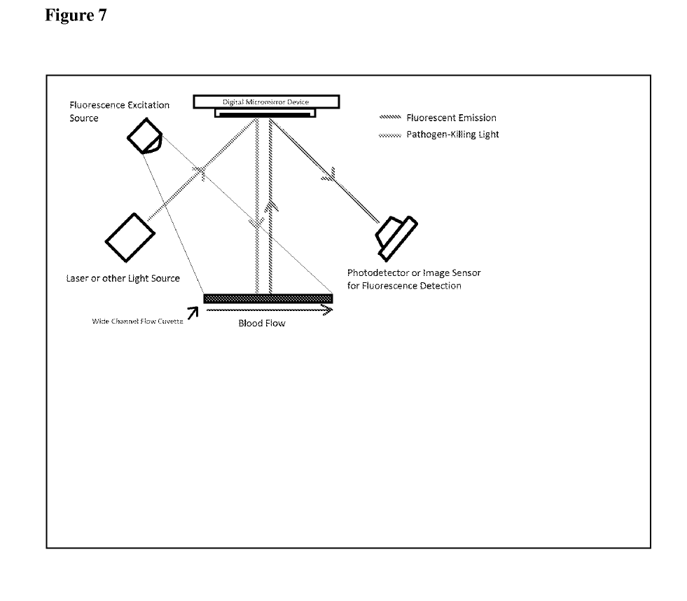

With no power applied to the DMD, the mirrors remain flat. When the DMD is powered, however, the mirrors can be individually set to either of two ‘states,’ (typically ±12° from flat), allowing light from a given source to be reflected in one of two different directions, based on digital image data. FIG. 5 illustrates a scanning electron microscope (SEM) image of a single mirror vectored to a given ‘state’.

Initial applications for the DMD included high-end projection television and theatre projection systems. However, the precise control of light, flexible architecture, reliability, and the relatively low cost associated with the technology resulted in applications in many other fields. DMDs have since been used extensively in medical imaging, mask-less photolithography, laser micro-machining, and even in 3D printing. This diversity of applications is a testament to the broad range of wavelengths and power sources supported by the technology.

DMDs support wavelengths from UV to the near infrared (NIR) regions of the electromagnetic spectrum. Supported light sources include simple light-emitting diodes (LEDs), high-power mercury and xenon lamps, and lasers.

In an embodiment of the present invention, the digital micromirror device is used in such a way as to facilitate high-speed, high-resolution fluorescence imaging of body fluids, and subsequent annihilation of pathogens detected by simultaneous projection of intense photonic energy to multiple detected pathogens.

Fluid, such as whole blood, cerebrospinal fluid (CSF), or plasma, containing fluorescently tagged pathogens is imaged under flowing conditions during a continuous dialysis, hemoperfusion, or other similar procedure. A wide-channel flow cuvette or similar device provides an ideal optical window for both detection and photo-annihilation of pathogens.

FIG. 4 shows an example of a flow cuvette that in an embodiment of the present invention. To achieve acceptable flow rates, the cuvettes can be scaled in size, or multiple channels can be implemented in a parallel processing manner.

FIG. 5 illustrates the basic elements of the system. For clarity, passive optical components that are typical to projection, sensing, and imaging systems are omitted from the illustration. Additional components, such as optical filters, collimating and shaping lenses, beam-splitters, and others devices, would be necessary in a practical system. The figure shows a centrally mounted DMD that would be used in both the sensing of fluorescently labeled pathogens and in the delivery of intense photonic energy to destroy those pathogens.

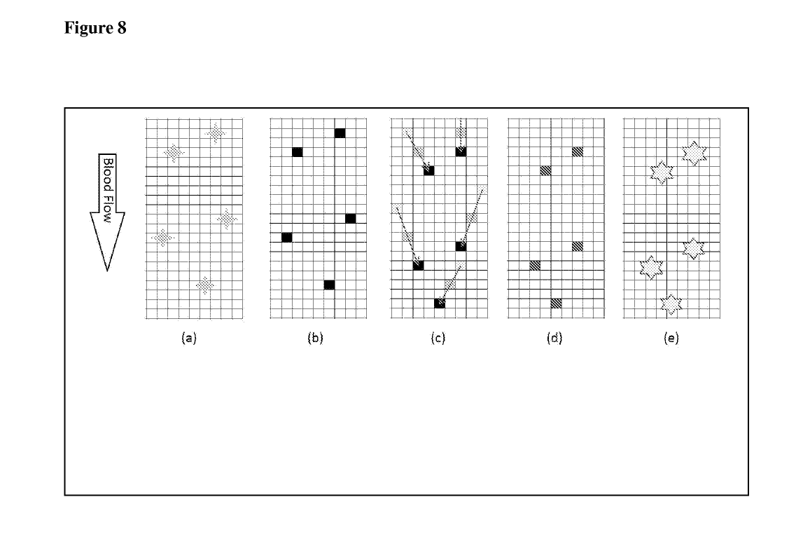

An independent (asynchronous) light source is used to continuously excite the fluorescent pathogens in the flowing blood or other body fluid. A repetitive process begins with the DMD configured for sensing. Mirrors are vectored so that light from fluorescence emission (observed at the flow cuvette) is routed to the photodetector or image sensor. After sufficient detection and processing time, the DMD is configured for projection. The micromirrors are re-positioned to route intense photonic energy from a laser or other intense light source back to the flow cuvette where it impinges on the detected pathogen targets and destroys them. The process would then continue in a “Sense, Process, and Annihilate” cycle as illustrated in FIG. 8.

At low resolution, this figure illustrates how the sensing window on the flow cuvette is discretized into a virtual grid based on the spatial resolution of the DMD. Information gained from the fluorescent sensing would comprise a digital bitmap image representing a location map of detected pathogens (FIG. 8(a,b)). Analysis of multiple sequential images, using image processing methods familiar to those skilled in the art, would allow identification and tracking of individual pathogens (FIG. 8(c)) and enable precise targeting for subsequent irradiation with the selected light source (FIG. 8(d,e)).

Embodiments of the present invention include:
1. A method for treating a body fluid containing a targeted infectious pathogenic antigen characterized by:
a. applying a treatment of a fluorescently tagged antibody to the body fluid in a first stage to form a fluorescently tagged antibody-targeted pathogenic antigen complex,
b. passing the fluorescently tagged antibody treated body fluid through a second stage,
c. detecting the fluorescently tagged antibody, and
d. eliminating the fluorescently tagged antibody-targeted pathogenic antigen from the body fluid in the second stage using a high-energy radiation emissive source.
2. The method above wherein the high-energy radiation emissive source is a laser.
3. The method above characterized by targeting a pathogenic antigen selected from the group consisting of bacteria, viruses, parasites, fungi, and prions.
4. The method above wherein the high-energy radiation emissive source is a laser, characterized by a first stage, comprising a first step including directing a first fluorescently tagged antibody against the targeted pathogenic antigen.
5. The method above further characterized by:
a. removing the bodily fluid from a patient to produce an extracorporeal bodily fluid; and
b. returning the bodily fluid to the patient after substantially removing the treatment in the second stage.
6. The method above further comprising adding medications to the bodily fluid prior to returning to the patient to further eliminate the targeted pathogenic antigen (bacteria, viruses, parasites, fungi, or prions).
7. The method above further comprising testing the bodily fluid for the efficacy of treatment before returning the extracorporeal bodily fluid to the patient.
8. A method for treating a bodily fluid characterized by:
a. applying a treatment consisting of a luminous antibody to the bodily fluid in a first stage to form a luminous antibody targeted antigen complex,
b. passing the luminous antibody treated body fluid through a second stage, and
c. detecting the luminous antibody, and
d. eliminating the luminous antibody antigen complex from the body fluid in the second stage using a high-energy radiation emissive source.
9. A method for treating a bodily fluid characterized by:
a. applying a treatment consisting of a luminous antibody to the bodily fluid in a first stage to form a luminous antibody targeted antigen complex,
b. passing the luminous antibody treated body fluid through a second stage, and
c. detecting the luminous antibody, and
d. eliminating the luminous antibody antigen complex from the body fluid in the second stage using a high-energy radiation emissive source is projected using a digital micromirror device.
10. A method for treating a bodily fluid characterized by:
a. applying a treatment consisting of a luminous antibody to the bodily fluid in a first stage to form a luminous antibody targeted antigen complex,
b. passing the luminous antibody treated body fluid through a second stage, and
c. detecting the luminous antibody, and
d. eliminating the luminous antibody antigen complex from the body fluid in the second stage using a laser projected using a digital micromirror device.

Numerous modifications and variations of the present invention are possible. It is, therefore, to be understood that within the scope of the following claims, the invention may be practiced otherwise than as specifically described. While this invention has been described with respect to certain preferred embodiments, different variations, modifications, and additions to the invention will become evident to persons of ordinary skill in the art. All such modifications, variations, and additions are intended to be encompassed within the scope of this patent, which is limited only by the claims appended hereto.

All documents, books, manuals, papers, patents, published patent applications, guides, abstracts and other references cited herein are incorporated by reference in their entirety. Other embodiments of the invention will be apparent to those skilled in the art from consideration of the specification and practice of the invention disclosed herein. It is intended that the specification and examples be considered as exemplary only, with the true scope and spirit of the invention being indicated by the following claims.

1. A method for treating a body fluid containing a targeted infectious pathogenic antigen characterized by:
a. applying a treatment of a fluorescently tagged antibody to the body fluid in a first stage to form a fluorescently tagged antibody-targeted pathogenic antigen complex,
b. passing the fluorescently tagged antibody treated body fluid through a second stage,
c. detecting the fluorescently tagged antibody, and
d. eliminating the fluorescently tagged antibody-targeted pathogenic antigen from the body fluid in the second stage using a high-energy radiation emissive source.

2. The method of claim 1 wherein the high-energy radiation emissive source is a laser.

3. The method of claim 1 characterized by targeting a pathogenic antigen selected from the group consisting of bacteria, viruses, parasites, fungi, and prions.

4. The method of claim 2, characterized by a first stage, comprising a first step including directing a first fluorescently tagged antibody against the targeted pathogenic antigen.

5. The method of claim 1 further characterized by:
a. removing the bodily fluid from a patient to produce an extracorporeal bodily fluid; and
b. returning the bodily fluid to the patient after substantially removing the treatment in the second stage.

6. The method of claim 5 further comprising adding medications to the bodily fluid prior to returning to the patient to further eliminate the targeted pathogenic antigen (bacteria, viruses, parasites, fungi, or prions).

7. The method of claim 5 further comprising testing the bodily fluid for the efficacy of treatment before returning the extracorporeal bodily fluid to the patient.

8. A method for treating a bodily fluid characterized by:
a. applying a treatment consisting of a luminous antibody to the bodily fluid in a first stage to form a luminous antibody targeted antigen complex,
b. passing the luminous antibody treated body fluid through a second stage, and
c. detecting the luminous antibody, and
d. eliminating the luminous antibody antigen complex from the body fluid in the second stage using a high-energy radiation emissive source.

9. A method for treating a bodily fluid characterized by:
a. applying a treatment consisting of a luminous antibody to the bodily fluid in a first stage to form a luminous antibody targeted antigen complex,
b. passing the luminous antibody treated body fluid through a second stage, and
c. detecting the luminous antibody, and
d. eliminating the luminous antibody antigen complex from the body fluid in the second stage using a high-energy radiation emissive source is projected using a digital micromirror device.

10. A method for treating a bodily fluid characterized by:
a. applying a treatment consisting of a luminous antibody to the bodily fluid in a first stage to form a luminous antibody targeted antigen complex,
b. passing the luminous antibody treated body fluid through a second stage, and
c. detecting the luminous antibody, and
d. eliminating the luminous antibody antigen complex from the body fluid in the second stage using a laser projected using a digital micromirror device.
